(19) United States
(12) Patent Application Publication
(10) Pub. No.: US 2025/0295881 A1
(43) Pub. Date: Sep. 25, 2025

(54) METHOD FOR CONCENTRATING OXYGEN INSIDE A MASK

(71) Applicant: Anil K. Tiwari, Corona del Mar, CA (US)

(72) Inventor: Anil K. Tiwari, Corona del Mar, CA (US)

(21) Appl. No.: 19/231,397

(22) Filed: Jun. 6, 2025

Related U.S. Application Data
(63) Continuation of application No. 17/172,969, filed on Feb. 10, 2021, now abandoned.

(51) Int. Cl. A61M 16/06; A61M 16/08; A61M 16/10

(52) U.S. Cl. CPC A61M 16/0672; A61M 16/085; A61M 16/107; A61M 2016/0661; A61M 2210/0618; A61M 2210/0625

(57) ABSTRACT

A system for concentrating oxygen inside a mask with filters is provided. The system comprising a mask body having a contoured shape that fits above the patient's nose and mouth; filters which allow gasses but not contaminants to pass through; a port with detachable cap for gas sampling; an inlet port connected which can be open to room air or attached to standard oxygen tubing or a ventilator circuit. The mask may be worn with or without an oxygen source. The mask body is firmly sealed against the patient's face with the filters functioning to prevent microbial contamination of the environment while concentrating oxygen within the lumen of the mask.

### CROSS-REFERENCE TO RELATED APPLICATION

This application claims benefit of U.S. patent application Ser. No. 17/172,969, filed Feb. 10, 2021, the disclosures of which are hereby incorporated by reference herein in their entirety.

### FIELD OF THE INVENTION

The present device and method is generally related to medical devices, and more specifically, to an oxygen mask with filters and gas sampling which is capable to be worn without an oxygen source or with an oxygen source via standard oxygen tubing or via a ventilator hose attachment.

### BACKGROUND

For medical and surgical procedures, patients often receive sedatives such as benzodiazepines, opioids, and propofol which cause respiratory depression that necessitates supplemental oxygen via an oxygen face mask with or without capnography monitoring.

In prior art, conventional oxygen masks comprise tent like structures which are strapped over the nose and mouth of the patient, often using an elastic band or bands behind the patient's head. Oxygen is supplied via tubing to the front of the mask which is then concentrated into the lumen of the mask body and inhaled. Conventional masks are able to concentrate oxygen within the lumen of the mask using small perforations which allow gasses to enter and exit the mask. Without supplemental oxygen delivered into the mask, the small perforations are often inadequate to allow a patient to breathe without the feeling of suffocation.

In the prior art, numerous attempts have been made to improve the function of oxygen retention inside the mask. Most recently, an approach to oxygen retention inside the mask is provided, where an oxygen reservoir on the inspiratory side with or without a one-way valve is provided between the reservoir and the mask. The reservoir fills with oxygen during exhalation and is available to meet inspiratory maximum flow requirements during inspiration. This approach makes the mask system bulky and complex. Moreover, this approach limits the usage of mask to a single application and different varieties of masks have to be used for different medical applications. Hence, none of the prior art shows a single mask for different users with variety of applications.

Therefore, it is desirable to provide an oxygen mask that facilitates the concentration or retention of adequate oxygen level inside the mask and that can be used along with standard oxygen tubing or with a ventilator circuit. Moreover, it is desirable to provide a universal mask with microbial filtration that can be used in various scenarios such as in procedure rooms, operating rooms, emergency rooms and on the field by paramedics.

In order to overcome the above-mentioned problems, the present invention provides an oxygen concentrating mask for facilitating the retention of adequate oxygen level inside the mask, filters which allow gases to enter and exit the mask, gas sampling, and the ability to be used with or without supplemental oxygen. The present oxygen mask includes a clear, soft and malleable plastic or silicone construction with an elastic strap that is used to secure onto the wearer's face. The present invention or mask comprises a front opening covered with a filter in addition to two side openings covered with filters. When the mask is not connected to supplemental oxygen or gas sampling, there are caps and filters to ensure each breath is filtered without the feeling of suffocation. The mask allows connection to a supplemental oxygen source or ventilator circuit in addition to providing gas sampling when needed.

### SUMMARY

This section provides a general summary of the disclosure and is not a comprehensive disclosure of its full scope or all of its features.

One aspect of the present invention provides an oxygen mask for concentrating oxygen in the mask, the face mask comprising: a mask body having a contoured shape that fits above the patient's nose and mouth; elastic strap or straps; gas-permeable side filters which provide microbial protection while concentrating oxygen within the mask; a sampling port to connect with gas sampling or capnography; an inlet port connected to an oxygen source for blowing oxygen into the mask; a front filter which reduces the work of breathing through the mask when used without supplemental oxygen; a removable cap which covers the sampling port when not used.

### BRIEF DESCRIPTION OF DRAWINGS

Further areas of applicability will become apparent from the description provided herein.

The skilled artisan will understand that the drawings are primarily for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings:

FIG. 1A and FIG. 1B shows the side view and the top view respectively of a mask with an ability to concentrate oxygen using filters in lieu of perforations, in accordance with an embodiment of the present invention.

FIG. 2 illustrates ports 112 fitted to the mask for gas sampling in accordance with an embodiment of the present invention.

FIG. 3 illustrates a view of the cap 306 to cover the sampling port 112 in accordance with an embodiment of the present invention.

FIG. 4 illustrates a front filter 400 of the mask in accordance with an embodiment of the present invention.

FIG. 5 illustrates a pair of side filters 104a and 104b of the oxygen mask in accordance with an embodiment of the present invention.

FIG. 6 illustrates the oxygen adaptor tubing adaptor 115 which fits onto connector 108 and allows the present invention to be attached to hospital oxygen supply tubing.

FIG. 7 illustrates the connector piece 108 which fits into the oxygen inlet 106 of the present invention.

FIG. 8 is a pictorial representation showing a graph of oxygen flow rate versus sampled exhaled oxygen flow rate showing the utility and evidence of design functionality in accordance with an embodiment of the present invention.

### DETAILED DESCRIPTION

In the following detailed description of embodiments of the invention, numerous specific details are set forth in order to provide a thorough understanding of the embodiments of invention. However, it will be obvious to a person skilled in art that the embodiments of invention may be practiced with or without these specific details. In other instances well known methods, procedures and components have not been described in detail, so as not to unnecessarily obscure aspects of the embodiments of the invention.

The terminology used herein is for the purpose of describing particular embodiments only and is not intended to be limiting of the invention. As used herein, the term “and/or” includes any and all combinations of one or more of the associated listed items. As used herein, the singular forms “a”, “an”, and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present invention discloses a mask for concentrating or retaining the oxygen at a titratable level inside the mask when worn by a wearer while also providing microbial filtration and gas sampling. The invention provides a mask with functions without an oxygen source, or with oxygen via oxygen tubing or ventilator hose that can be used for variety of applications and can be used in procedure rooms, operating rooms, emergency rooms and on the field by paramedics.

The mask is made of clear malleable plastic or silicone construction with an elastic strap that is used to secure onto a patient's face. The mask comprises a countered shape to fit around the patient's nose; a front inlet for oxygen covered with a filter; two side openings covered with filters; sampling ports; caps and connectors.

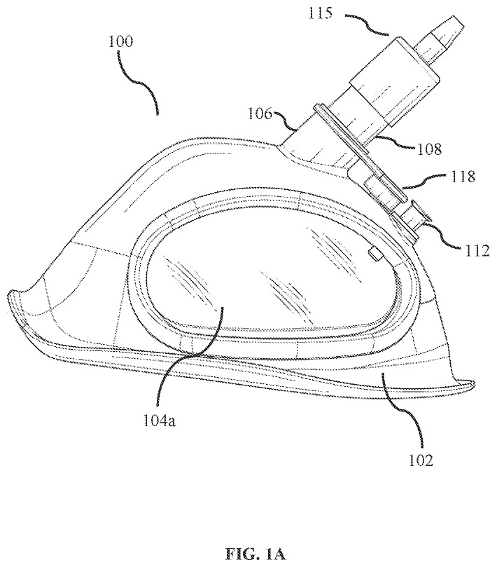

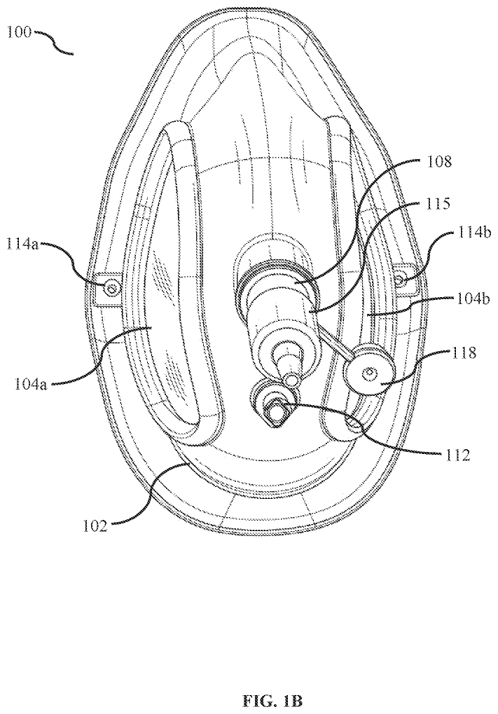

FIG. 1A and FIG. 1B show side and the top views respectively of a mask 100 with an ability to concentrate oxygen inside the mask with filters in accordance with an embodiment of the present invention. Referring to FIG. 1A-B, the mask 100 comprises a mask body 102 defining a countered shape to fit around the wearer's nose, an inlet port 106 for directing the flow of gases to the interior of the mask 100, two side filters 104a and 104b allow the wearer to breathe with minimal effort while serving to concentrate oxygen and filter microbial contaminants, strap attachment slots 114a and 114b to secure the ends of an elastic strap for securing the mask around the wearer's head, port 112 for gas sampling, gas sampling cap 112, connector 115 for attaching to standard oxygen tubing.

The inlet port 106 of the mask 100 allows the vent assembly 108 and front filter 400 to be attached, effectively filtering breath when used without supplemental oxygen. When supplemental oxygen is required, port 106 will attach to a ventilator circuit or oxygen tubing can be attached with connector piece 115. The connector 115 is securely snap-fit with the second end of the vent assembly 108. The connector 115 terminates in a conduit coupler to attach to standard hospital oxygen tubing.

The mask body 102 is generally molded of a low flammability, gas-impermeable material, such as non-toxic medical grade plastic polymer or silicone material. The mask body 102 material can be transparent to allow clinicians or health care personnel to observe the patient's or wearer's mouth and nose in addition to condensation inside of the mask. The mask body 102, connections and attachments thereto may be disposable.

The mask body 102 defines a cavity adapted to fit over the mouth and the nose of the wearer. The peripheral edge of the mask body 102 is contoured so as to substantially seal against the surrounding facial tissue of the wearer to establish an inner chamber portion or inner-space. The peripheral edge can be of any shape as long as it is contoured so as to substantially seal against the surrounding facial tissue of the patient or normal wearer.

The mask body 102 can be held to the wearer by an attachment mechanism. Any suitable mechanism can be utilized. The mechanism can include an elastic material and non-elastic material. The attachment mechanism can include clips, buttons, clamps, hook and loop (e.g., Velcro®), the like, etc. Preferably, the attachment mechanism can be a two piece (or more) partially elastic passive/active adjustable/detachable strap system which may or may not attach via holes 114a and 114b in FIG. 1B.

In some embodiments of the present invention the perimeter lining of mask 100 can be lined with a gas permeable or semi-permeable material or filters to inhibit an inner-space of the mask body from substantial contamination with room air when the oxygen mask is in use. The perimeter lining can be made of various materials including, but not limited to, cushion, padding, foam, and elastic. The perimeter lining may be formed integrally and unitarily with the mask body 102 or may be formed separately and permanently joined to the mask body. The perimeter lining may be thinner than the other areas of the mask body.

The mask 100 comprises an inlet port 106 for directing a flow of gas to the interior of the mask 100. The inlet port 106 is formed on the top of the mask body 102 i.e. around nose and mouth. The inlet port 106 allows oxygen to flow from an oxygen source to the inner-space of the mask 100. For an adult patient in the present invention, the oxygen flow rate can be varied from 0-10 liters/min. Since the peripheral edge of the mask body 102 is firmly sealed against the surroundings and the non-porous filters (two side filters and a front filter) prevent the oxygen from escaping from the mask in order to retain the adequate oxygen level inside the mask 100. The sampling port 112 of the mask 100 is covered by the cap 118 in order to prevent the escaping of gas from the mask when sampling port 112 is not needed for gas analysis. Given the large inlet port 106 and filters 104a and 104b, mask 100 provides the ability to concentrate oxygen inside the mask while allowing the patient to breathe effortlessly with or without supplemental oxygen supply.

The mask 100 described herein can be used in applications where it is desirable to reduce contaminants flowing to and from a wearer's nose and mouth during exhalation and inhalation. Such contaminants can include, for example, bacteria, viruses, surgical smoke, and the like. As used herein, “wearer”, “user” and “patient” can be synonymous. Generally, the mask described herein may be used by health care professionals for patients to avoid spreading contaminants from their breathe into sterile environments or to other health care workers. The mask 100 of the present invention works as a surgical mask for patients in sterile operating or procedure rooms.

The filters 104a and 104b are disposed on both sides (i.e. left side and right side) of the mask body 102 to allow the patient to breathe with minimum effort. The oxygen mask 100 of the present invention may serve as a surgical mask to a patient for reducing the risk of contaminating the sterility of an operating or procedure room and when needed equipped via oxygen via vent assembly 108 or connector 115. The filters 104a and 104b are made of materials including but is not limited to, paper, polypropylene, polyethylene, polyester, and/or ePTFE.

The filters 104a, 104b and front filter (not shown in figure) may be formed integrally with the molded mask body 102 or may be inserted into the mask body 102. The filters are attachable or connectable as accessories in various ways. For example, polytetrafluoroethylene (PTFE) filter 104 attaches to the mask body composed of silicone or plastic such as but not limited to Polyvinyl chloride (PVC). The two side filters and a front filter do not have pours or vents in order to prevent the wearer's breath from contaminating the environment while also retains adequate oxygen level inside the mask.

The PTFE (fine powder resin) is expanded into a 3-dimensional web-like structure which creates billions of microscopic pores. This structure utilizes the inherent hydrophobic (water-resistant) and non-stick nature of PTFE to allow removal of particulate captured on the membrane surface. Hence, it can block dust, water droplets, micro-organisms, etc. In the preferred embodiment, the filters 104a, 104b and the front filter 400 are sintered PTFE filter, ePTFE. Polyethylene, polypropylene and or polyester blends.

In another aspect of the present invention the inlet port 106 is formed right above the half-way line that separates the upper half and the bottom half of the mask body 102. In this manner, the inlet port 106 is located around the nostrils of the patient when the mask 100 is worn allowing oxygen to be delivered to the nasal area.

The mask 100 also comprises a sampling port 112 designed to connect to gas sampling tubing for gas sampling or capnography for sampling exhaled breath or an expiratory gas from a wearer or patient. A cap 118 is used to cover the sampling port 112 when gas sampling is not being utilized. The cap 118 is designed to cover the opening of port 112 to prevent the oxygen/gas from escaping from the mask in order to retain the oxygen concentration inside the mask at an adequate level and prevent microbial contamination.

In the preferred embodiment the sampling port or outlet port 112 is positioned at the front of the oxygen face mask 100 and between the left side filter 104a right side filter 104b. Preferably, the outlet port 112 is positioned below the inlet port 106 and between the left side filter 104a and right side filter 104b (as shown in FIG. 1B).

The sampling port 112 may be connected to gas sampling tubing that is coupled to a device or sensor for sampling and/or analyzing an expiratory gas or exhaled gas of the oxygen mask 100. The gas may be sampled from the gas sampling tubing or a component present with the gas may be sampled. A sampled gas may contain other component(s) such a therapeutic nebulized or aerosolized component or agent. A gas may be expired gas. An expired gas may be mixed, in part, with delivered oxygen, or room air before sampling. In one example, a gas may not contain expired air (e.g., if the patient is not breathing). In one example, carbon dioxide is sampled (capnography). In another example, oxygen is sampled. In another example, end tidal partial pressure of the gas (e.g., carbon dioxide) may be measured (or otherwise determined or calculated).

In another aspect of the present invention, ventilator circuit and tubing may be attached to the inlet port 106 for supplemental gas flow of oxygen and or air at varying concentrations. When ventilator tubing is not available, standard hospital oxygen tubing may be attached via connector 115.

FIG. 2 illustrates sampling port 112 fitted to the mask 100 in accordance with an embodiment of the present invention. Referring to FIG. 2, the sampling port 112 is embedded, molded or wedged into mask 100 for gas sampling. sampling port 112 consists of two ends: inside end which is male and outside end which is female to attach to gas sampling tubing.

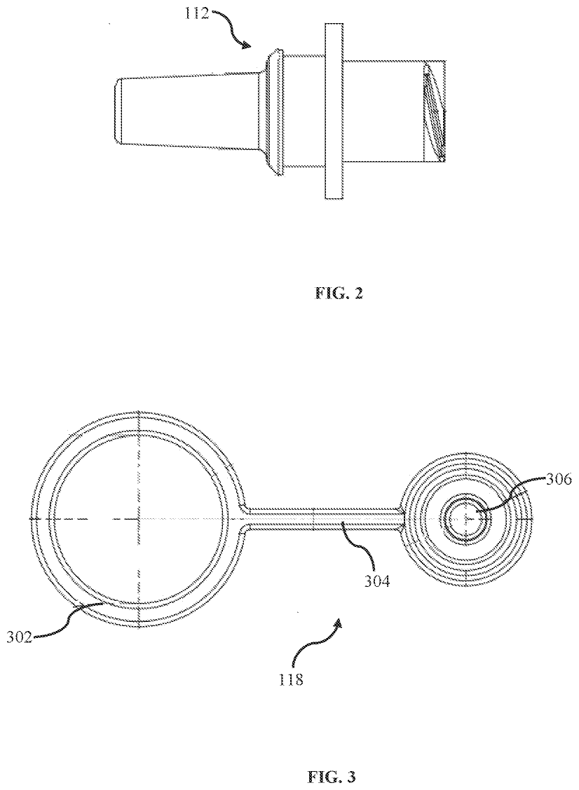

FIG. 3 illustrates a cap 118 to cover the sampling port 112 in accordance with an embodiment of the present invention. Referring to the FIG. 3, the cap 118 comprises a cylindrical plug 306, an attachment strip 304 and a holding ring 302. The cap 118 is used to cover the sampling port 112 by the user or health care professional when sampling port 112 is not utilized. When the cap 118 is placed on the sampling port 112, the cylindrical plug 306 is snap fitted inside the inner perimeter of the sampling port 112. The holding ring 302 is designed to fit around the diameter of vent assembly 108 and sandwiched against the opening 106. The attachment strip 304 connects holding ring 302 with cap 118. The complete structure of cap 118 is made up of a plastic material.

FIG. 4 illustrates the front filter 400 of the face mask in accordance with an embodiment of the present invention. Referring to the FIG. 4, the front filter 400 has protruding flanges designed to be sandwiched, snap-fitted, molded or embedded between the vent assembly 108 and the inside diameter of opening 106. The front filter 400 is made up of sintered PTFE, ePTFE. Polyethylene, polypropylene and or polyester blends.

The front filter 400 configured to prevent the wearer's breath from contaminating the environment when connector 115 is not connected.

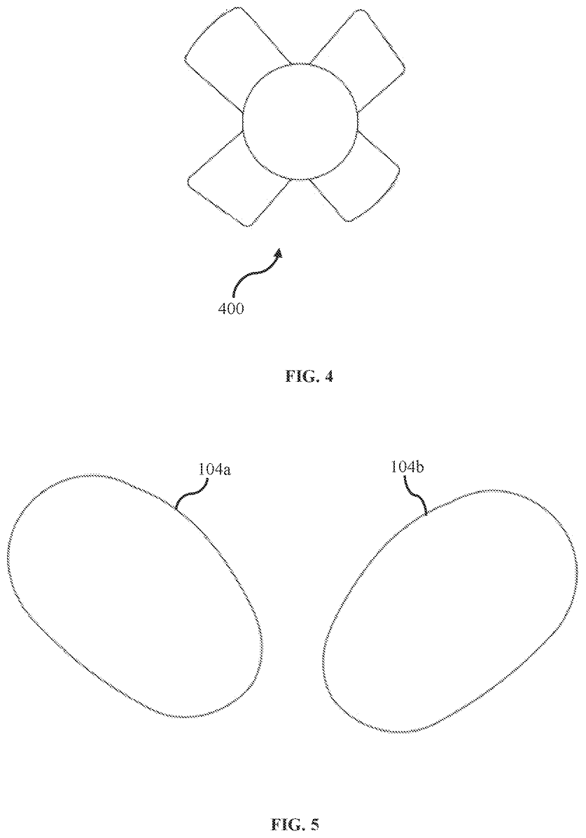

FIG. 5 illustrates a pair of side filters 104a and 104b of the oxygen mask in accordance with an embodiment of the present invention. Referring to FIG. 5, both the side filters 104a and 104b may be formed integrally with the molded mask body 102 or may be inserted into the mask body 102.

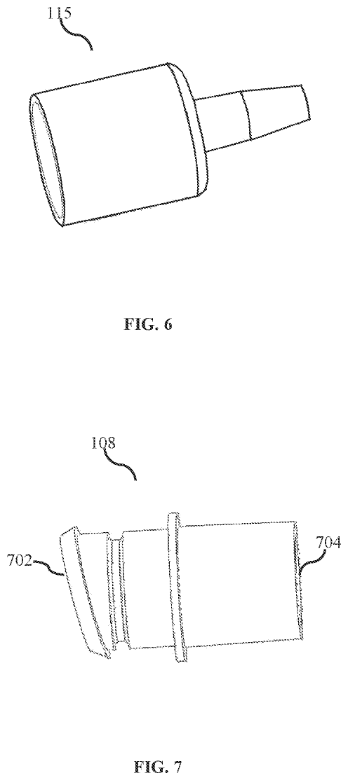

FIG. 6 shows an exemplary view of the connector 115 for attaching the ventilator circuit or standard oxygen tubing in accordance with an embodiment of the present invention. The connector 115 is securely snap-fit with the second end 704 (as shown in FIG. 7) of the vent assembly 108. The connector 115 terminates in a conduit coupler to attach to standard hospital oxygen tubing, when supplemental oxygen is required.

FIG. 7 shows the detailed view of the vent assembly 108 in accordance with an embodiment of the present invention. The vent assembly 108 consists of two ends illustrating as first end 702 and second end 704. The first end 702 of the vent assembly 108 is securely fitted with the inlet port 106. The holding ring 302 is designed to fit around the diameter of first end 702 of vent assembly 108 and sandwiched against the opening of inlet port 106. The front filter 400 is sandwiched, snap-fitted, molded or embedded between the first end 702 of the vent assembly 108 and the inside diameter of opening of inlet port 106. The second end 704 of vent assembly 108 is used for snap fitting of the connector 115.

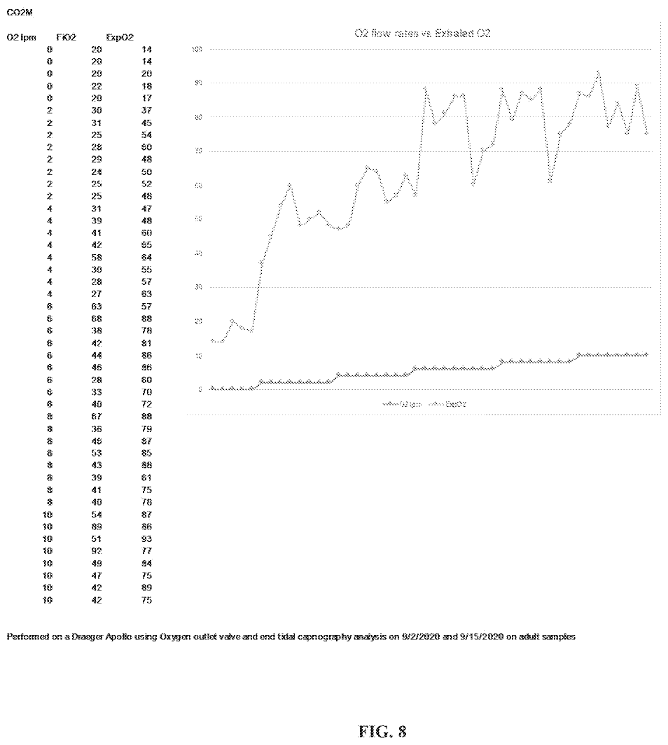

Proof of concept and utility of the present invention is demonstrated in FIG. 8 as collected data and graph show oxygen flow rate versus exhaled oxygen concentration. The oxygen flow rate can be varied from 0-10 liters/min using an oxygen supply source and tubing via connector 115. Exhaled gas was sampled via sampling port 112. The data was collected using Draeger Apollo gas sampling on adult subjects using the oxygen outlet valve (to 115) and end tidal capnography analysis via 112. Referring to FIG. 8, the graph shows different values of exhaled oxygen rate at given oxygen flow rate and demonstrates the efficacy of the present invention with filters in providing concentrated oxygen under various flow rates.

Fraction of inspired oxygen (FᵢO₂) is the molar or volumetric fraction of oxygen in the inhaled gas. Patients with respiratory compromise or receiving sedation are provided with oxygen-enriched air, which means a higher-than-atmospheric FᵢO₂. Natural air includes 21% oxygen, which is equivalent to FᵢO₂ of 0.21. Oxygen-enriched air has a higher FᵢO₂ than 0.21; up to 1.00 which means 100% oxygen. FᵢO₂ is typically maintained below 0.5 even with mechanical ventilation, to avoid oxygen toxicity but there are applications when up to 100% is routinely used. Often used in medicine, the FᵢO₂ is used to represent the percentage of oxygen participating in gas-exchange.

A single oxygen mask 100 incorporating connectors according to various aspects of the present invention can function as a surgical face mask with no supplemental oxygen, a low (or simple) oxygen mask, a medium oxygen mask, a high oxygen (i.e. from 30% to 90% oxygen concentrations) mask, obviating the need for multiple masks and thereby resulting in cost savings.

For additional details relating to the present invention, materials and manufacturing techniques of the level of ordinary skill in the art can be used. The same may be true for aspects based on the method of the present invention with respect to additional actions commonly or logically used.

Also, optional features of the described variations of the invention can be described and claimed independently or in combination with any one or more of the features described herein. Similarly, a reference to a singular element includes the possibility that there are pluralities of the same element. More specifically, the singular form (“a,” “and,” “said,” and “the”) is not expressly required by the context as used herein and in the appended claims. As long as it includes a plurality of instructions. It is further noted that the claims may be drafted to exclude optional elements.

Therefore, this statement should serve as a preceding basis for the use of exclusive terms such as “simply”, “only”, etc. or “negative” limitation in connection with the description of the elements of the claims is intended. Unless defined otherwise herein, all technical and scientific terms used herein have the same meaning as commonly understood by one of ordinary skill in the art to which this invention belongs. The scope of the invention is not limited by this specification, but only by the plain meaning of the terms used in the claims.

What is claimed is:

1. A mask for respiratory use, comprising:
a mask body having a contoured shape configured to cover the nose and mouth of a patient, the mask body comprising a pair of lateral openings;
an inlet port positioned on the front portion of the mask body;
a vent assembly coupled to the inlet port, the vent assembly comprising a primary filter element disposed across the inlet port;
a pair of side filter elements, each removably positioned within a respective one of the lateral openings;
wherein the inlet port is configured for connection with a supplemental oxygen source, and the pair of side filter elements are configured to allow passive air exchange when the supplemental oxygen source is not connected; and
wherein the pair of side filters and the filter on the inlet port prevent microbial contamination from the patient's breath, both with or without the use of supplemental oxygen.

2. The mask of claim 1, further comprising a gas sampling port positioned adjacent to a nose region of the mask body.

3. The mask of claim 2, wherein the gas sampling port includes a cap to cover the gas sampling port when the gas sampling port is not utilized to prevent leakage of respiratory gas and contaminants.

4. The mask of claim 1, wherein the inlet port includes a connector adapted to receive a standard hospital oxygen tubing or a ventilator circuit.

5. The mask of claim 1, wherein the vent assembly is made of a hard plastic material.

6. The mask of claim 1, wherein the vent assembly comprises a rigid housing made of medical-grade plastic.

7. The mask of claim 1, wherein the oxygen source delivers oxygen at a variable flow rate of 0-10 liters/min.

8. The mask of claim 1, wherein the mask body is sealed at its periphery by a medical-grade foam or elastomeric rim to inhibit leakage during use.

9. The mask of claim 1, wherein the side filter elements and the primary filter element are configured to block microbial or viral particles during inhalation and exhalation.

10. The mask of claim 1, wherein the side filters are removably secured within the side openings using a snap-fit or friction-fit configuration.

11. The mask of claim 1, wherein the filter on the inlet port is a replaceable medical-grade particulate filter.

12. The mask of claim 1, wherein the inlet port is positioned on a front-central portion of the mask body and is oriented to direct oxygen flow toward the nose of the patient.

13. The mask of claim 1, wherein the side filters are adapted to provide filtration of exhaled air when supplemental oxygen is not supplied through the inlet port.

14. The mask of claim 1, wherein the mask body includes an internal chamber defined by the shape and contour of the mask body above the nose and mouth of the patient.

15. The mask of claim 1, wherein the mask body includes a flexible peripheral rim configured to form a seal with the patient's face to reduce leakage.

16. The mask of claim 1, wherein the inlet port includes a locking ring or threaded interface to secure the filter and prevent displacement during oxygen delivery.

17. The mask of claim 1, wherein the mask further comprises a pair of adjustable elastic straps configured to secure the mask to the patient's head.

18. The mask of claim 1, wherein the filter elements on the inlet port and side filters are disposable after a single use.
